(19) United States
(12) Patent Application Publication
(10) Pub. No.: US 2012/0264197 A1
(43) Pub. Date: Oct. 18, 2012

(54) H2S Removal from Contaminated Gases

(75) Inventor: Michael J. Mitariten, Pittstown, NJ (US)

(73) Assignee: Guild Associates, Inc., Dublin, OH (US)

(21) Appl. No.: 13/087,596

(22) Filed: Apr. 15, 2011

(51) Int. Cl. A61L 9/01; C12M 1/00

(52) U.S. Cl. 435/266; 435/283.1

(57) ABSTRACT

A process for removing hydrogen sulfide from a raw natural gas stream such as biogas from landfills or controlled anaerobic digestion comprises passing the natural gas stream though a separation unit such as a PSA unit to form a product stream comprising a high concentration of methane and a low pressure tail gas containing hydrogen sulfide, passing the tail gas through a biofilter which includes bacteria that degrades the hydrogen sulfide to sulfur and sulfate compounds which are washed from the biofilter. The tail gas stream subsequent to treatment in the biofilter can be flared to the atmosphere without significant SOx emissions.

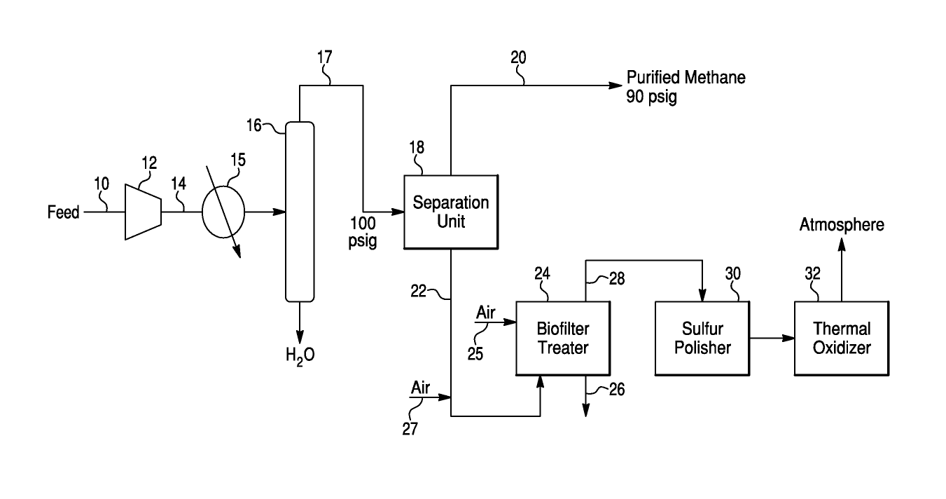

### FIELD OF THE INVENTION

This invention relates to an improved method of H₂S removal from contaminated gas streams with focus on digester feeds and landfill gases (collectively “biogas”).

### BACKGROUND OF THE INVENTION

This invention relates to the processing of biogas with methane components into a natural gas stream suitable for injection into pipeline gas.

Many biogas assets exist in the United States. Landfills are a prime example of biogas as it is of relatively low quality and flow rate. Landfill gas (LFG), as produced naturally by anaerobic digestion of accumulated wastes, is nominally a 55/45 mixture of methane and carbon dioxide, with trace contaminants of siloxane compounds, hydrogen sulfide and a number of volatile organic compounds (VOC's). The LFG collection is aided by the operation of blowers that create a negative pressure in the landfill. This negative pressure causes air infiltration into the LFG stream, especially at the periphery of the landfill where gas wells exist primarily to reduce the migration of LFG (and its corresponding odors) to nearby properties. This air infiltration introduces nitrogen and oxygen into the LFG. This contaminated gas stream is difficult to process successfully to produce a gas that can be injected into natural gas pipelines.

In some instances, LFG has been processed and accepted into pipelines at qualities less than normally required. Unless flows in the pipeline accepting lower quality processed gas render its contaminant contribution insignificant, this lower quality gas can cause difficulties for natural gas end users. Improper or less than optimal treatment of the LFG may result in a carryover of landfill gas contaminants into the pipeline and eventually into businesses or residences. Complete treatment of landfill and other biogases will provide additional indigenous and sometimes renewable resources for the well-developed natural gas distribution systems in the United States. Landfill sites also have a major advantage in that they are often located near larger metropolises and corresponding high gas usage areas. Recovery of the uncontaminated methane for use in normal natural gas markets will result in a more efficient use of the energy content than the more usual use of LFG for electrical generation, and its attendant energy conversion losses.

The safe disposal of waste or contaminating materials has been recognized as a significant health and economic issue for many years. The ability to merely dump raw materials into the oceans or landfills is no longer an acceptable mechanism for disposal of the waste. Waste organic matter including that found in raw wastewater (i.e., sewage), sludge from sewage treatment facilities, farm waste, organic industrial waste, leachate, and so forth is a principle cause of water pollution. Therefore, waste organic matter from these and other sources ideally is treated before release into the environment in order to reduce or eliminate the presence of environmentally harmful organic compounds.

One method of treating waste organic matter, especially in wastewater treatment plants and concentrated animal farms, is through anaerobic digestion. Anaerobic digestion is the biological degradation of organic material without oxygen present in which bacteria degrade or digest or decompose the organic matter fed into the system. The anaerobic digestion process has been utilized to treat and remove organic compounds from waste products such as sewage, sewage sludge, chemical wastes, food processing wastes, agricultural residues, animal wastes, including manure and other organic waste and material. As is well known, organic waste materials are fed into an anaerobic digestion reactor or tank which is sealed to prevent entrance of oxygen and in these airfree or “anoxic” conditions, anaerobic bacteria digests the waste. Anaerobic digestion may be carried out in a single reactor or in multiple reactors of the two-stage or two-phase configuration. Heat is normally added to the reactor or reactors to maintain adequate temperatures for thermophilic or mesophilic bacteria which accomplish the breakdown of the organic material. Mixing of the wastes by either mechanical or gas recirculation can be provided to accelerate digestion.

The tremendous increase in demand for natural gas in recent years has made the gas producers far more dependent on “sour” gas fields than ever before. As used herein, a “sour” gas is defined as a gas containing mercaptans and/or hydrogen sulfide. Landfill gas and gas obtained from organic and biological waste known as digester feeds contain unacceptable levels of hydrogen sulfides and are considered to be “sour” gas fields. “Sweetening” is defined as the removal of the mercaptans and hydrogen sulfide from a gas or liquid stream. Typical pipeline limits on H₂S are 4 ppm and due to the cost of removing H₂S, formerly, when a gas well came in “sour”, it was often capped off because the supply and demand situation did not permit its purification. Recently, however, these capped wells have been put into production and are being utilized regardless of their hydrogen sulfide and mercaptan content.

Several methods for sweetening hydrocarbons streams have been proposed and utilized in the past, including both chemical and physical techniques.

A prior art system for removing hydrogen sulfides from biogas is the iron sponge method of purifying natural gas, utilizing iron oxide impregnated wood chips in a packed bed. The gaseous mixture containing hydrogen sulfide and/or mercaptans contacts a packed bed of iron oxide sponge, preferably chemically absorbing the sulfur impurities on the exposed iron oxide surface. A major disadvantage of this method of sweetening natural gas is that the fusion of iron sponge particles with sulfur frequently causes a high pressure drop through the bed. Moreover, the operational cost is high because the iron sponge adsorbent is not regenerable. When the useful life thereof has been reached the spent absorbent must be removed and replaced. Finally, the iron sulfide is pyrophoric and thus presents serious problems with the disposal of the used iron oxide. Such a non-regenerable system can be attractive for smaller flow rates or small concentrations of H₂S. However, higher levels of H₂S or higher flow rates lead to ongoing and high cost for the frequent absorbent replacement.

A widely used chemical system for treating natural gas streams involves scrubbing with amine solvents. The natural gas is passed through the amine solution which absorbs the hydrogen sulfide. The solution from the absorption equipment is passed to a stripping column where heat is applied to boil the solution and release the hydrogen sulfide. The lean, stripped solution is then passed to heat exchangers, and returned to the absorption equipment to again absorb hydrogen sulfide gas. The principle disadvantages of the amine system are its high operating cost, the corrosive nature of the absorbing liquid, its inability to remove mercaptans and water from gas streams, as well as its limited ability to selectively remove hydrogen sulfide from carbon dioxide.

Pressure swing adsorption is a well-known method for the separation of bulk gas mixtures and for the purification of gas streams containing undesirable impurities. Gas separations by pressure swing adsorption (PSA) are achieved by coordinated pressure cycling of a bed of adsorbent material which preferentially adsorbs at least one more readily adsorbable component present in a feed gas mixture relative to at least one less readily adsorbable component present in the feed gas mixture. That is, the bed of adsorbent material is contacted with a ready supply of a feed gas mixture. During intervals while the bed of adsorbent material is subjected to the ready supply of feed gas mixture and the bed is at or above a given feed pressure, a supply of gas depleted in the at least one more readily adsorbable component may be withdrawn from the bed. Eventually, the adsorbent material in the bed becomes saturated with the at least one more readily adsorbable component and must be regenerated. At which point, the bed is isolated from the ready supply of feed gas mixture and a gas enriched in the at least one more readily adsorbable component is withdrawn from the bed, regenerating the adsorbent material. In some instances, the bed may be subjected to a purge with depleted gas to facilitate the regeneration process. Once the adsorbent material is sufficiently regenerated, the bed is again subjected to the ready supply of feed gas mixture and depleted gas can once again be withdrawn from the bed once the pressure on the bed is at or above the given feed pressure. This cycle may be performed repeatedly as required.

The use of PSA systems for the removal of impurities, such as nitrogen and carbon dioxide, from natural gas streams are well known and used in the purification of natural gas streams. In general, an effective PSA process for the removal of nitrogen from natural gas, described in U.S. Pat. No. 6,197,092, issued Mar. 6, 2001, involves a first pressure swing adsorption of the natural gas stream to selectively remove nitrogen and produce a highly concentrated methane product stream. Secondly, the waste gas from the first PSA unit is passed through a PSA process which contains an adsorbent selective for methane so as to produce a highly concentrated nitrogen product. One important feature is the nitrogen selective adsorbent in the first PSA unit. The adsorbent is a crystalline titanium silicate molecular sieve adsorbent and is based on ETS-4, which is described in U.S. Pat. No. 4,938,939. Adsorbents having controlled pore sizes are referred to as CTS-1 (contracted titano silicate-1) and are described in U.S. Pat. No. 6,068,682, issued May 30, 2000. The CTS-1 molecular sieve is particularly effective in separating nitrogen and acid gases selectively from methane. Due to the ability of the ETS-4 compositions, including the CTS-1 molecular sieves for separating gases based on molecular size, these molecular sieves have been referred to as Molecular Gate® sieves.

There are also numerous patents that describe PSA processes for separating carbon dioxide from methane or other gases. One of the earlier patents in this area is U.S. Pat. No. 3,751,878, which describes a PSA system using a zeolite molecular sieve that selectively adsorbs CO₂ from a low quality natural gas stream operating at a pressure of 1000 psia, and a temperature of 300° F. The system uses carbon dioxide as a purge to remove some adsorbed methane from the zeolite and to purge methane from the void space in the column. U.S. Pat. No. 4,077,779, describes the use of a carbon molecular sieve that adsorbs CO₂ selectively over hydrogen or methane. After the adsorption step, a high pressure purge with CO₂ is followed by pressure reduction and desorption of CO₂ followed by a rinse at an intermediate pressure with an extraneous gas such as air. The column is then subjected to vacuum to remove the extraneous gas and any remaining CO₂. The preferred type of adsorbent is activated carbon, but can be a zeolite such as 5A, molecular sieve carbons, silica gel, activated alumina or other adsorbents selective of carbon dioxide and gaseous hydrocarbons other than methane.

As noted above, in the removal of impurities from biogas feed stocks a wide variety of contaminants can be encountered. The primary contaminant is carbon dioxide and along with the CO₂ the feed commonly contains water vapor, hydrogen sulfide, nitrogen and oxygen, siloxanes and a variety of VOCs. The present assignee has provided a PSA system targeted at biogas. A typical application compresses the feed stock to 100 psig after which the compressed feed is cooled and temperature controlled and routed to the PSA system which adsorbs the impurities at typically 100 psig and with high quality enriched methane produced at 90 psig. Upon regeneration, which is typically under vacuum, the previously adsorbed impurities desorb from the adsorbent and are available as a low pressure tail gas stream.

The methane concentration of the tail gas stream is typically quite low with the actual concentration dependent upon the concentration of methane in the raw gas and the associated methane recovery rate of the PSA system. Typically, the methane concentration of the tail gas ranges between 10 and 30% and, thus, is of a low quality and often routed to a flare or a thermal oxidizer. Due to the fact that the tail gas will contain H₂S when flared, sulfur is emitted to the atmosphere. Environmental limits may exist on the amount of sulfur that can be emitted into the atmosphere. In such a case, the H₂S would need to be removed in some other form.

Due to the challenge of H₂S in biogas streams, the market has looked for lower cost means to handle the removal of higher quantities of H₂S. One technology which has been proven successful in sweetening biogas streams is the use of a biofilter. Biofilters are not true filtration units but are systems that combine the basic processes of absorption, adsorption, desorption and degradation of gas phase contaminants. Typical biofilters employ microorganisms affixed to organic media such as compost or peat. Extensive study into the growth properties of microorganisms (e.g., bacteria) in recent years has shown that particular types of bacteria may exist in complex forms comprising layers that tenaciously adhere to surfaces. Upon adhering to a surface, these complex forms of bacteria are termed “biofilms.” Generally, biofilms are comprised of sessile bacteria, this particular type of bacteria contributing to their inherent tenacity. As the contaminated air passes through the organic media, the contaminants sorb onto the biofilm and are biodegraded by the microorganisms. Biofilters usually employ water to humidify the contaminated gas stream prior to entry into the biofilter and to add nutrients for the microorganisms. If humidification proves inadequate, direct irrigation of the bed may be employed. Over time, all conventional media compacts, necessitating replacement.

A biotrickling filter uses inorganic material, such as diatomaceous earth, ceramic, or glass beads, for its packed bed. A biological fixed film grows on this bed. Water is sprayed on top of the packed bed and contaminated air is fed counter-currently or co-currently. In such a biofilter, the biogas containing H₂S passes, typically upward, through a bed of media in which acidophilic bacterial colonies are grown and used to remove the H₂S. As the biogas comes in contact with the biofilter, H₂S is solubilized and then subsequently oxidized by the microbes. Sulfur and sulfate compounds are formed as byproducts, which are typically purged with blow down water from the system. Thus, by using the bacteria based technology, a relatively low capital cost and continuous operating system can be used to remove relatively large volumes of H₂S.

However, since the biofilter reactor needs make up oxygen, commonly in the form of air, there is a major debit when using such a biofilter sweetening solution when the aim is to produce a product stream of enriched methane as substitute natural gas. In the biofilter sweetening process, oxygen, typically from air is required to be injected in the column in order to maintain the required oxygen concentration for optimized biological activity. While pure oxygen could be injected, more commonly air is injected into the stream. When air is injected, some of the oxygen is consumed in the biofilter treater and the nitrogen passes through and is present with the resulting biogas along with any unreacted oxygen. Thus, for applications producing pipeline quality methane gas from biogas, the use of a biofilter sweetening solution will result in the contamination of the biogas with nitrogen and unreacted oxygen. Consequently, placing the biofilter sweetening unit upstream of the PSA system (or other biogas upgrading technology) requires that the technology either has the ability to segment or remove the unconsumed oxygen and nitrogen or a lower quality product gas will be produced which may not meet pipeline requirements. For example, typical pipeline requirements limit nitrogen to 3 to 4% and while oxygen specifications vary widely, it is common to find requirements of less than 0.2% in a pipeline natural gas specification.

### SUMMARY OF THE INVENTION

A useful PSA system for upgrading biogas of this invention takes the approach of adsorbing H₂S when removing the CO₂, water vapor and other impurities from biogas feed stocks. The product gas from the PSA system is essentially free of H₂S and meets the requirement for pipeline quality gas which typically has a specification that the product can contain no more than about 4 ppm of H₂S.

Upon regeneration of the adsorbent in the PSA unit, the impurities, including the H₂S, are rejected at low pressure into the tail gas stream. The tail gas from the PSA is generally flared, though it can be used for fuel if a high enough methane concentration exists. In accordance with this invention, since the PSA system can remove H₂S from the biogas, a biofilter sweetening system can be placed on the tail gas from the PSA unit. Because the tail gas from the PSA is not routed to the pipeline, it is acceptable to inject oxygen, commonly in the form of air, into this tail gas stream and, thus, provide the oxygen as needed in the biofilter sweetening system. The biofilter sweetening system would then remove the H₂S with the sweetened gas being available to a flare or thermal oxidizer while dramatically reducing the emission of SOx to the atmosphere. It is important to note that in this placement of the biofilter sweetening unit on the tail gas stream, that the introduction of air is no longer an issue since this gas is not sent to the pipeline.

In its broadest aspect, this invention is directed to the treatment of a contaminant waste gas stream by a biofilter for removing hydrogen sulfide from the waste gas. Thus, any separation system which will remove contaminants such as CO₂, H₂S, VOCs, etc. from methane contained in a biogas stream can be used. Such separation systems include the PSA system as described above as well as membrane separation and solvent based systems including amines, physical solvents and water wash systems.

### BRIEF DESCRIPTION OF THE DRAWINGS

The FIGURE is a schematic of the process of this invention for removal of H₂S from biogas.

### DETAILED DESCRIPTION OF THE INVENTION

Methane is a primary constituent of landfill gas (LFG) and a potent contributor to greenhouse gasses. Municipal Solid Waste Facilities (MSWFs) are the largest source of human-related (anthropogenic) methane emissions in the United States, accounting for about 25 percent of these emissions in 2004. Additionally, these escaping LFG emissions are a lost opportunity to capture and use a significant energy resource. Substantial energy, economic, and environmental benefits are achieved by capturing LFG prior to release, which subsequently reduces greenhouse gasses. LFG capture projects improve energy independence, produce cost savings, create jobs, and help local economies. LFG is currently extracted from landfills using a series of wells and a vacuum system that consolidates the collected gas for processing. From there, the LFG is used for a variety of purposes including motor vehicle fuel, generator fuel, biodiesel production, natural gas supplement, as well as green power and heating.

Currently, MSWFs bury waste in layers over time. The basic structure is a floor and sidewalls of compacted clay, covered with a HDPE polymer liner, filled with layers of waste alternated with clay or soil layers. Once a landfill has reached a certain capacity, methane recovery wells are installed and gas is extracted from decay and decomposition of waste layers. As the waste body increases in height, non-apertured “riser pipe”, “casing”, “riser”, or “vertical pipe” is added to the existing extraction well. These terms may be used interchangeably for the tubular members extending into the waste body. Once the waste body reaches the design height or capacity it is covered with compacted soil, topsoil, or possible liner material and subsequently replanted with natural vegetation and left to decompose. LFG is created as the organic fraction of solid waste decomposes in a landfill, due to the process of methogenesis. LFG gas consists of about 50 percent methane, about 40-49% percent carbon dioxide, and a small amount of non-methane compounds as discussed above, including hydrogen sulfide. Landfills must be monitored over time to ensure that LFG emissions, groundwater leachate, and waste from the solid waste unit are not being released and impacting the environment. Methane extraction and recovery captures LFG and prevents emission of these air contaminants. Methane is first produced in the older, lower decomposing waste bodies. Subsequent layers produce methane at different times and rates. Currently, to extract methane from subsequent layers, wells are drilled to a desired depth or elevation and methane extracted. As decomposition continues shallower and shallower wells are required to reach gasses trapped in upper waste bodies. Multiple wells, pipe, equipment and repeated drilling are required to collect and transport the gas to the collection facility. LFG extraction, recovery and use is a reliable and renewable fuel option that represents a largely untapped and environmentally friendly energy source at thousands of landfills in the U.S. and abroad.

Capture of LFG can be used to produce electricity with engines, turbines, microturbines, or other technologies, used as an alternative to fossil fuels, or refined and injected into the natural gas pipeline. Capturing and using LFG in these ways can yield substantial energy, economic, environmental, air quality, and public health benefits. Internationally, significant opportunities exist for expanding LFG recovery and use while reducing harmful emissions.

Methane gas is also produced in controlled anaerobic digestion processes utilized to treat and remove organic compounds from waste products such as sewage, sewage sludge, chemical wastes, food processing wastes, agricultural residues, animal wastes, including manure and other organic waste and material. Organic waste materials are fed into an anaerobic digestion reactor or tank which is sealed to prevent entrance of oxygen and in these airfree or “anoxic” conditions, anaerobic bacteria digests the waste. Anaerobic digestion may be carried out in a single reactor or in multiple reactors of the two-stage or two-phase configuration. See, S. Stronach, T. Rudd & J. Lester, Anaerobic Digestion Processes in Industrial Wastewater Treatment, 1986, Springer, Verlag, pp. 93-120 for single reactors and pp. 139-147 for multi-stage operations. The products or effluent from anaerobic digestion consist of: (1) a gas phase containing methane, carbon dioxide, and smaller amounts of other gases, such as hydrogen sulfide, which in total comprise what is commonly called biogas; (2) a liquid phase containing water, dissolved ammonia nitrogen, nutrients, organic and inorganic chemicals; and (3) a colloidal or suspended solids phase containing undigested organic and inorganic compounds, and synthesized biomass or bacterial cells within the effluent liquid.

Methods for the anaerobic digestion or treatment of sludge, animal waste, synthesis gas or cellulose-containing waste are disclosed in, among others, U.S. Pat. No. 5,906,931 to Nilsson et al., U.S. Pat. No. 5,863,434 to Masse et al., U.S. Pat. No. 5,821,111 to Grady et al. U.S. Pat. No. 5,746,919 to Dague et al., U.S. Pat. No. 5,709,796 to Fuqua et al., U.S. Pat. No. 5,626,755 to Keyser et al., U.S. Pat. No. 5,567,325 to Townsley et al., U.S. Pat. No. 5,525,229 to Shih, U.S. Pat. No. 5,464,766 to Bruno, U.S. Pat. No. 5,143,835 to Nakatsugawa et al., U.S. Pat. No. 4,735,724 to Chynoweth, U.S. Pat. No. 4,676,906 to Crawford et al., U.S. Pat. No. 4,529,513 to McLennan, U.S. Pat. No. 4,503,154 to Paton, U.S. Pat. No. 4,372,856 to Morrison, U.S. Pat. No. 4,157,958 to Chow, and U.S. Pat. No. 4,067,801 to Ishida et al. These patents disclose different processes and equipment for the bioconversion, either by microbial digestion or enzymatic conversion, of those materials into methane and other useful materials.

The equipment used for the anaerobic digestion of waste into a biogas, which contains methane, varies greatly and is generally tailored to specific applications, which is known by one skilled in the art. Equipment that is suitable for a first type of feedstock generally has to be modified before it can be used for a second different type of feedstock.

The anaerobic microbe used in the anaerobic digester is any anaerobic bacterium, fungus, mold or alga, or progeny thereof, which is capable of converting the feedstock to a useful material in the anaerobic digester of the invention. Anaerobic microbes can be isolated from decaying or composted feedstock, can be endogenous to the area in which the feedstock was first obtained, and can be obtained from bacterial or fungal collections such as those of the American Type Culture Collection (ATCC) or have been genetically altered or engineered to convert a feedstock to a useful material.

The conditions inside the anaerobic digester will vary according to the useful material being produced, the anaerobic microbe being used, the configuration of the anaerobic digester, the feedstock being converted, the desired productivity of the anaerobic digester, and the form of microbe (immobilized or free-flowing) used. Immobilized microbes can be prepared using any methods known by the artisan of ordinary skills in the arts. The conditions used to culture the anaerobic microbe and maintain it viable in the anaerobic digester can be varied. Conditions which can be controlled include solids content, reaction solution composition, temperature, gas content, digestion rate, anaerobic microbe content, agitation, feed and effluent rates, gas production rate, carbon/nitrogen ratio of the feedstock, pressure, pH, and retention time in the digester, among other things.

Pressure swing adsorption (PSA) technology has recently found application for upgrading the biogas generated from land fills generated by MSWFs and controlled anaerobic digestion to a high-BTU fuel that can be sold directly to the pipeline or converted to CNG or LNG. The PSA process splits the biogas feed into two streams, a high-BTU product stream and a low-BTU tail gas stream consisting largely of carbon dioxide, moisture, hydrogen sulfide, other contaminants, and low levels of methane. In this invention, the tail gas stream is treated to remove hydrogen sulfides so that the remaining gas can be flared without producing SOx emissions that pollute the atmosphere.

The FIGURE illustrates a treatment system including a PSA unit that upgrades the biogas from a landfill or anaerobic digester. It should be understood that the process of this invention can be used to upgrade any natural gas stream containing H₂S. In the process of this invention, a feed 10 of biogas containing about 50-70% methane with the balance being carbon dioxide, hydrogen sulfide and other impurities such as, water, VOCs, siloxanes among other components is directed to a compressor 12, with or without precooling. Compressor 12 compresses the biogas stream 10 to the appropriate operating pressure of about 100 psig and produces a compressed biogas stream 14. The compressed biogas stream 14 is then cooled in condenser 15 to condense a portion of the water in the biogas stream, as shown by separator 16. The remaining gas is sent to the PSA (and vacuum pump) unit 18 via line 17. The PSA unit 18 contains a selective gas adsorbent as known in the art. The adsorbent used in PSA unit 38 is any known adsorbent or mixtures of adsorbents which adsorb the non-methane components of the biogas stream. Adsorbent materials suitable for use in the PSA unit 18 include, but are by no means limited to, activated carbon; carbon molecular sieve (CMS) adsorbents; activated alumina; silica gels; zeolites; and the titanium silicates. One skilled in the art is able to select a given adsorbent material or mixtures thereof, for use with a given feed gas mixture and desired product materials. The PSA unit 18 produces a high-quality methane stream, i.e. at least about 70% methane, for pipeline quality, at least about 90% methane, by selectively adsorbing much of the carbon dioxide, hydrogen sulfide, and other contaminants over the less readily adsorbable methane in the biogas stream. The high-quality methane output stream 20 is discharged at one end of the PSA unit 18. The PSA unit 18 also delivers a low pressure output stream 22 containing the desorbed impurities (carbon dioxide, hydrogen sulfide, and other contaminants) from the adsorption beds, which is generally referred to as “tail gas”. The composition of the tail gas 22 contains much of the carbon dioxide, hydrogen sulfide, and other contaminants from the biogas feed stream 10. While the majority of methane is contained in the high-quality methane product stream 20, the PSA unit 18 also adsorbs a portion of the methane from the biogas feed 10 which is then contained in tail gas 22 during the desorption of the PSA unit 18. However, because the concentration of methane in tail gas 22 is often too low to provide an adequate heating value to be useful as a fuel, tail gas 22 requires disposal in a flare. If tail gas 22 was flared untreated, the hydrogen sulfide would be converted to SOx. Most municipalities have regulations concerning the amount of SOx that is to be emitted into the atmosphere. Accordingly, flaring the untreated tail gas stream 22 is problematic.

The PSA process is of itself a well-known means of separating and purifying a less readily adsorbable gas component contained in a feed gas mixture of said component with a more readily adsorbable second component, considered as an impurity or otherwise. Adsorption commonly occurs in multiple beds of a solid adsorbent at an upper adsorption pressure, with the more selectively adsorbable second component thereafter being desorbed by pressure reduction to a lower desorption pressure. The beds may also be purged, at pressures above or below that of atmospheric pressure and typically at such lower pressure for further desorption and removal therefrom of said second component, i.e., the removal of impurities with respect to a high purity product gas, before repressurization of the beds to the upper adsorption pressure for the selective adsorption of said second component from additional quantities of the feed gas mixture as the processing sequence is carried out, on a cyclic basis, in each bed in the PSA system. Such PSA processing is disclosed in the Wagner patent, U.S. Pat. No. 3,430,418, and in the Fuderer et al. patent, U.S. Pat. No. 3,986,849, wherein cycles based on the use of multi-bed systems are described in detail. Such cycles are commonly based on the release of void space gas from the product end of each bed, in so called cocurrent depressurization step(s), upon completion of the adsorption step, with the released gas typically being employed for pressure equalization and for purge gas purposes. The bed is thereafter countercurrently depressurized and/or purged to desorb the more selectively adsorbed component of the gas mixture from the adsorbent and to remove such gas from the feed end of the bed prior to the repressurization thereof to the adsorption pressure.

The PSA system can be operated with at least one, and typically at least two adsorbent beds, as may be desirable in the given applications, with from three to about 12 or more adsorbent beds commonly being employed in conventional practice.

While the PSA system produces a high-BTU fuel that can be sold directly to the pipeline or converted to CNG or LNG, the low-BTU tail gas produced by the PSA system is often too low in heating value to be useful as a fuel and requires disposal. The present invention is a method for disposing the tail gas in an environmentally and economically efficient manner.

While the invention has been described as using a PSA system to remove the non-methane contaminants from the biogas feedstream, it is possible to use other separation systems alone, or in addition to, the PSA system described. Thus, in the FIGURE, PSA unit 18 can instead, or in addition to, be a membrane separation unit or a solvent based system including amines, physical solvents and water wash units. In a membrane separation unit, the non-methane contaminants such as CO₂, H₂S and VOC's are permeated through the membrane and become waste gas stream 22. The methane is retained by the membrane and can be removed from the member separation unit via line 20.

In general, the membrane separation unit removes carbon dioxide and H₂S gas from the crude biogas stream by use of a bundle of hollow fibers disposed within the membrane separation unit. The surface of each hollow fiber is made from a membrane material which may be readily permeated by carbon dioxide gas, hydrogen sulfide, oxygen gas, water vapor, and certain VOC gases. The membrane material is substantially less permeable to methane. In one hollow-fiber configuration as the crude biogas stream flows through the membrane separation unit, it initially travels inside of the hollow fiber bundles. However, substantial amounts of carbon dioxide gas, H₂S, oxygen gas, water vapor, and VOC gases permeate the membranes of the fibers. This permeate gas may be collected as the waste gas stream 22. The methane and other gases which do not permeate through the membranes of the fibers may be separately collected as the product gas stream 20. This product gas can be further treated, if necessary, in another separation unit such as the PSA unit described above.

During this process, the membrane separation unit is typically operated at an inlet pressure of from about 175 to about 225 psi and at a temperature of from about 100 to about 135° F.

An example of a suitable membrane separation unit for use in accordance with the present disclosure is the BIOGAZ membrane system, available from Air Liquide—Medal of Newport, Del.

Still further, another separation system which can be used is what is known as a water wash system in which the biogas feedstream 10 is washed with water. The water wash absorbs the non-methane contaminants from the biogas feedstream. These contaminants can be separated from the water using separation tanks to form the waste gas stream 22, which will contain CO₂, hydrogen sulfide, VOC's, etc.

In accordance with the present invention, the tail gas stream 22 is treated to remove the hydrogen sulfide therefrom, prior to the tail gas being flared to the atmosphere. Thus, as shown in the FIGURE, tail gas stream 22 is directed to a biofilter 24 which is capable of converting the hydrogen sulfide in the tail gas stream to sulfur or sulfate compounds which can be removed from the tail gas. The exact type or configuration of the biofilter is not critical to this invention, as long as such biofilter is capable of degrading hydrogen sulfide and removing the hydrogen sulfide from the tail gas stream. Thus, biofilters which employ microorganisms affixed to organic media such as compost in which the useful type of bacterial for degrading hydrogen sulfides are added and adhere to the organic media surface in the form of biofilms. As the contaminated tail gas passes through the organic media, the contaminants such as hydrogen sulfide sorb onto the biofilm and are degraded by the microorganisms. Biofilters known as biotrickling filters which use inorganic or other synthetic organic material to support the biofilms can also be utilized and, are preferred. The support medium is typically in the form of a packed bed and the bacteria grows within the packed bed as a biological fixed film. Particular useful biotrickling filters are produced by Biorem, Victor, N.Y. The description below refers to a particular biotrickling filter developed by Biorem, but it is to be understood that other types of biofilters can be utilized so long as the hydrogen sulfide is degraded and removed from the tail gas stream.

According to the FIGURE, a particularly useful biofilter is an aerobic biotrickling filter designed in a forced-draft, up flow configuration. The tail gas from line 22 enters the base of a tower 24 and then passes up through the support media contained as a packed bed in tower 24. The support media provides an ideal environment for the establishment of a biofilm consisting of acidophilic bacterial colonies with adequate void volume for free gas flow. The media in the packed bed and the biofilm which is formed thereon by the bacteria are kept moistened by way of recirculated water. As the tail gas stream comes in contact with the biofilm, hydrogen sulfide is solubilzed and subsequently oxidized by the microbes. Sulfur and sulfate compounds are formed as by-products and are purged with the recirculated water blown down the tower and exits via line 26. A continuous flushing design is utilized to virtually eliminate downtime enabling by-product removal while the system is in operation. A small volume of air is injected into the process directly into tower 24 or tail gas stream 22 via lines 25 and 27, respectively to maintain the required oxygen concentration in the gas for optimized biological activity. Previous to this invention, the use of such biofilters to sweeten landfill gas and the like would be problematic since the addition of air into the landfill feedstream added additional contaminants, oxygen and nitrogen, which would have to be removed downstream. In this invention, the biofilter is added to treat the tail gas and, as such, the high purity product methane stream leaving the PSA unit 18 via line 20 is not contaminated with the oxygen and nitrogen from the air. The waste gas now treated in the biofilter 24 leaves tower 24 via line 28 substantially free of hydrogen sulfide. This gas can be then flared to the atmosphere without emitting excessive amounts of SOx pollutants.

In those municipalities or locations, where air standards may be very strict and the emissions of SOx into the atmosphere severely limited, further treatment of the tail gas stream 28 may be warranted prior to flaring to the atmosphere. Thus, the FIGURE also illustrates an optional sulfur treating system 30 which can include, for example, the iron sponge particles discussed previously, the Sulfa treat media, a product of M-I SWACO Corporation, believed to be an iron-based absorbent and carbon, all of which chemically absorb any sulfur impurities still contained in the treated tail gas stream 28. Inasmuch as the amount of hydrogen sulfide which still remains in tail gas stream 28 is minimal, the sulfur polishing media does not have to be removed and replaced as frequently as if this material were used to remove the hydrogen sulfide directly from the biogas feed.

In some embodiments according to the present disclosure, the system may also include the thermal oxidizer unit 32. In particular, if the initial crude landfill gas stream 10 includes a substantial amount of VOCs, these VOCs may also be present in the waste gas stream 22. In these instances, it is preferred that the system include the thermal oxidizer 32, which is in flow communication with the biofilter 24 and, if used, sulfur polishing system 30, so as to receive the treated waste gas stream 22, and substantially destroy the VOCs therein.

In general, the use of the thermal oxidizer unit 32 in the system is preferred if the concentration of VOCs in the waste gas stream 22 is sufficiently high to necessitate treatment prior to release of the gas into the atmosphere. The thermal oxidizer unit 32 preferably destroys at least about 98 percent of the VOCs present in the waste gas stream. With the level of VOCs so reduced, the treated waste gas stream 22 may be released into the atmosphere. As the term is used herein the thermal oxidizer can also be a flare.

The thermal oxidizer unit 32 is preferably sized in accordance with the flow rate of the waste gas stream and the amount of combustible components to be treated. The thermal oxidizer unit 32 is typically operated at a pressure close to atmospheric and at a system design temperature at which the desired VOC destruction ratios are achieved. Typically, the temperature is at least about 1500° F.

Since the biofilm treater may operate at a pressure lower than required by the thermal oxidizer, a blower (not shown) may be added after the biofilm treater. If the methane concentration of the tail gas is such that it is desired to be used as fuel, either directly or by blending with a higher heating value stream, the tail gas can be used as fuel rather than sent to a thermal oxidizer or flared to the atmosphere. The eventual use of the gas after treatment to remove H₂S is not critical to the invention.

### Example

A digester feed stream is compressed to 100 psig and with the conditions below is treated in a PSA system to yield the following material balance:

Design Material Balance after Compression:

| | Feed | Product | Tail Gas |
|---|---|---|---|
| Flow, SCFM | 1150 | 634 | 516 |
| Pressure, psig | 100 | 90 | 2 |
| Temperature, F. | 100 | 100 | 150 |
| Composition, | | | |
| Mol % | | | |
| CH4 | 60.00 | 98.00 | 13.36 |
| CO2 | 39.80 | 2.00 | 86.19 |
| H2S | 0.20 | Nil | 0.45 |
| H2O | Saturated | Dry | By Difference |

After the methane product is removed the tail gas is treated in a biofilm treater to provide the separation below:

| | | Treated | Treated gas |
|---|---|---|---|
| | | Gas after | after Sulfur |
| | Tail Gas | Biofilm | Polisher |
| Flow, SCFM | 516 | 514 | 514 |
| Pressure, psig | 2 | <2 | <2 |
| Temperature, F. | 150 | 100 | 100 |
| Composition, | | | |
| Mol % | | | |
| CH4 | 13.36 | 13.42 | 13.42 |
| CO2 | 86.19 | 86.58 | 86.58 |
| H2S | 0.45 | 50 ppm | 4 ppm |
| H2O | By | Saturated | Saturated |
| | Difference | | |

H2S removed 2.3 SCFM

1. A process of treating a raw natural gas stream which contains non-methane contaminants including hydrogen sulfide comprising; passing said raw natural gas stream though a separation unit, for forming a product stream having a higher concentration of methane than in said raw natural gas stream and a waste stream containing said non-methane contaminants including hydrogen sulfide, passing said waste stream though a biofilter which contains a film of bacteria on a support medium, said bacteria capable of degrading said hydrogen sulfide into sulfur and sulfate compounds removed from said waste stream to form a treated waste gas, adding an oxygen-containing gas stream to said biofilter to maintain bacterial activity, said treated waste gas removed from said biofilter having a concentration of hydrogen sulfide substantially less than said waste gas entering said biofilter.

2. The process of claim 1, wherein said raw natural gas stream is a biogas stream taken from a landfill or controlled anaerobic digestion of organic waste.

3. The process of claim 2, wherein said separation unit is a PSA unit which includes an adsorbent selective for said non-methane contaminants, said PSA unit forming a non-adsorbed product stream having a higher concentration of methane than in said raw natural gas stream and a lower pressure waste stream containing said non-methane contaminants, said raw natural gas stream being pressurized to at least about 100 psig for entry into said PSA unit.

4. The process of claim 3, wherein the pressurized natural gas stream is cooled to condense water from said raw natural gas stream prior to entry into said PSA unit.

5. The process of claim 1, wherein said biofilter is a biotrickling filter in the form of a tower having a top and bottom and having contained therein a medium to support said film of bacteria and allow gas to pass therethrough, said waste gas being passed from the bottom of said filter upwardly through the medium and out from the top of said tower as a treated waste gas, passing water from the top of said tower down through said medium to wash and carry sulfur and sulfate compounds from said biofilter.

6. The process of claim 1, wherein said contaminants further include carbon dioxide and VOCs and said waste stream contains said carbon dioxide and VOCs.

7. The process of claim 1, wherein said oxygen-containing gas is air which is added directly into said biofilter or into said waste gas stream or both.

8. The process of claim 1, wherein said oxygen-containing gas is an enriched oxygen stream which is added directly into said biofilter or into said waste gas stream or both.

9. The process of claim 1, wherein water is periodically introduced into said biofilter to wash said biofilm to remove said sulfur and sulfate compounds from said biofilter.

10. The process of claim 1, wherein said waste gas stream subsequent to treatment in said biofilter is passed in contact with a sulfur absorbent material so as to remove further hydrogen sulfide from said treated waste gas.

11. The process of claim 10, wherein said sulfur absorbent material contains iron.

12. The process of claim 10, wherein the said sulfur absorbent material is activated carbon.

13. The process of claim 1, wherein said waste gas leaving said biofilter is flared to the atmosphere.

14. The process of claim 1, wherein said waste gas leaving said biofilter is used as fuel, with or without blending with a higher heating value stream.

15. The process of claim 10, wherein the waste gas leaving said sulfur absorbent material is flared to the atmosphere.

16. The process of claim 6, wherein the waste gas leaving said biofilter is thermally oxidized to degrade said VOCs.

17. The process of claim 10, wherein the waste gas stream leaving the biofilter is treated with a sulfur absorbent to further remove hydrogen sulfide contaminants and said waste gas leaving the sulfur absorbent is thermally oxidized to degrade VOCs.

18. The process of claim 3, wherein said non-adsorbed product stream contains at least 70 mol % methane.

19. The process of claim 3, wherein said non-adsorbed product stream contains at least 90 mol % methane.

20. An apparatus for treating a raw natural gas stream which contains contaminants, including hydrogen sulfide comprising;
a PSA unit which includes an adsorbent selective for non-methane contaminants, said PSA unit forming a non-adsorbed product stream having a higher concentration of methane than in said raw natural gas stream and a lower pressure waste stream containing said non-methane contaminants, including hydrogen sulfide,
means to direct said waste stream to a biofilter,
a biofilter for receiving said waste stream and containing a film of bacteria on a support medium, said bacteria capable of degrading said hydrogen sulfide in to sulfur and sulfate compounds, which are removed from said waste stream to form a treated waste gas,
means to add an oxygen-containing gas stream to said biofilter to maintain bacterial activity,
means to direct said treated waste gas from said biofilter.
